(19) United States
(12) Patent Application Publication
(10) Pub. No.: US 2004/0002670 A1
(43) Pub. Date: Jan. 1, 2004

(54) Wound care apparatus and methods

(75) Inventors: James M. Mothersbaugh, Akron, OH (US); James L. Canterbury, Munroe Falls, OH (US); Detlev F. Smith, Fairlawn, OH (US)

Correspondence Address: Hahn, Loeser + Parks LLP, Twin Oaks Estate, 1225 W. Market Street, Akron, OH, 44313, US

(73) Assignee: Circle Prime Manufacturing, Inc.

(21) Appl. No.: 10/360,821

(22) Filed: Feb. 7, 2003

Related U.S. Application Data
(60) Provisional application No. 60/355,370, filed on Feb. 7, 2002.

(51) Int. Cl. A61F 5/00

(52) U.S. Cl. 602/1

(57) ABSTRACT

The invention is directed to wound care apparatus and methods that provide for non-contact wound covering. The apparatus can be configured to be positioned on any portion of the body, and comprises a body member having a base providing a sealing surface for application to the skin or other surface. The body member has a shape and size to at least substantially circumscribe the area of a wound. The body member further includes at least one wall forming an enclosed space over the wound area. The apparatus substantially seals a wound under the enclosed space from the external environment. One or more ports to allow the ingress or egress of fluids or treatment agents may be provided. Treatment methods are also provided for treatment of wounds using the device.

### TECHNICAL FIELD

The invention relates to wound care apparatus and methods relating to a non-contact wound treatment apparatus and methods for wound care, wherein the wound treatment apparatus is attached to an area of a patient's body peripheral to a wound, and forming an enclosed space extending over the wound area. The apparatus and methods may be used to effectively seal the wound from the external environment, and in an embodiment, allows for the ingress and egress of fluids for the treatment and care of a wound.

### BACKGROUND OF THE INVENTION

In the care of wounds, a variety of dressings have been utilized which require intermittent, routine changing by a skilled individual to ensure proper healing of the wound. Traditional wound dressings, such as bandages, cover and directly contact the wound, which can actually interfere with the healing process. Bandage type dressings typically do not isolate the wound from the external environment or infectious agents that may be present, thereby inhibiting proper healing, or being detrimental to the healing process.

Further, various dressings which directly contact the wound, the dressing may actually cause repetitive pressure injuries, as for supplied by the dressing to the wound is distributed directly on the wound itself. Bandage type dressings also do not allow visualization of the wound area without removal of the dressing, again requiring a painful and time-consuming process. Due to the problems associated with bandage type wound dressings, there is a need to provide a wound dressing which avoids the problems associated with bandage type dressings or other dressings which directly contact the wound area, as well as provide for unique capabilities in conjunction with wound care treatment processes.

A problem has also been encountered with respect to properly dressing wounds, which are of a unique character or are uniquely positioned on the patient's body. As an example, if there is an interruption in the integrity of the chest wall of a patient, air is allowed to fill the pleural space, which eliminates the negative pressure needed to inflate the lung. Typical wound dressings would not be effective to avoid this problem, as the dressing is designed to “breathe” or allow the exchange of gases therethrough.

Also in a variety of environments, the protection and treatment of wounds, such as in field situations, poses a significant problem for medical practitioners. As an example, in military operations, wound dressings are particularly problematic, due to the possibly severe nature of the environment in which the wound dressing is to be applied, particularly in the situations commonly dealt with in today's military activities. An issue is presented as to how to properly protect a soldier or other person having a wound in an environment where nuclear, biological, or chemical weapons may have been or may be used. In such environments, military personnel typically wear MOPP suits, which are designed to protect the individual from chemical and/or biological weapons and nuclear fallout. If the MOPP suit is penetrated by a projectile or the like, causing a wound to the individual, the integrity of the MOPP suit is destroyed. In such a circumstance, care for the wound as well as care for the patient by maintaining the integrity of the MOPP suit becomes important.

Also, in possibly contaminated environments, it may be advantageous for personnel to protect wounds from the environment prior to entering it even when wearing a protective suit, such as a MOPP suit. Additionally, once the personnel have been exposed to a contaminated environment, they must be decontaminated in a “shuffle pit” or by other decontamination procedures. In these situations, typical wound dressings would have to be removed prior to decontamination, and it would be worthwhile to enable a wound dressing to remain in its protective position in this situation.

In the treatment of wounds, it has also been found that various treatment modalities may enhance healing of the wound, such as by the flow of hydrated fluids or hyperberic oxygen over the wound area. Alternatively, wound care may comprise treating the wound using a solution of saline, antibiotic solutions, haemostatic solution to stop bleeding of an acute wound, enzymatic debriding agents, heated gases or other fluids. Other forms of wound treatment may comprise the use of light, such as low frequency infrared radiation or the application of suction to the wound area to remove fluids and/or stimulate healing.

It would therefore be advantageous to provide a wound dressing which permits various wound care modalities to be performed. It would also be advantageous to allow the monitoring of variables related to the wound, such as temperature, pH, the presence of certain agents or byproducts or the like.

### SUMMARY OF THE INVENTION

Based upon the foregoing, it is an object of the invention to provide wound care apparatus and methods that overcome the limitations of the prior art, and provide significant advantages for the dressing and treatment of wounds in a variety of circumstances and/or environments.

It is a further object of the invention to provide wound care devices and methods, which provide for non-contact wound covering, configured to be positioned on any portion of the body.

A further object of the invention is to provide wound care devices and methods which allow various treatment modalities to be employed in conjunction with the apparatus to enhance or promote wound healing.

Yet another object of the invention is to provide a wound care apparatus and methods which allow dressing of a wound as well as closure of a breach in the integrity of external articles of clothing or the like.

The invention in general is directed to a wound treatment apparatus comprising a body member configured to have a base providing a sealing surface for application to a surface. The sealing surface of the body member is configured to have a shape and size to at least substantially circumscribe the area of a wound. The body member further comprises at least one wall extending upwardly from the sealing edge, forming an enclosed space between the at least one wall and the surface to which the sealing edge is applied. The sealing surface is adapted to be applied and retained on the skin surface to substantially seal a wound under the enclosed space from the external environment.

The invention is also directed to a body member of a type as previously described, but also comprising at least one ingress and/or egress from the enclosed space formed by the at least one wall.

Other aspects, objects and advantages of the invention will become apparent upon a reading of the detailed description of various embodiments, in conjunction with the drawings.

### BRIEF DESCRIPTION OF THE DRAWINGS

FIG. 1 is a front view of a first embodiment of a wound care apparatus according to the invention.

FIG. 2 is a top view of the wound care apparatus as shown in FIG. 1.

FIG. 3 is a cross sectional view of the apparatus as shown in FIG. 1.

FIG. 4 is an elevational view of an alternative embodiment of the apparatus.

FIG. 5 is an enlarged, partial sectional view of an outlet port associated with the embodiment of FIG. 4.

FIG. 6 is a cross sectional view of the embodiment as shown in FIG. 4, having plugs used in combination therewith.

FIGS. 7a-7c schematically show alternative embodiments of the invention for use on various portions of a patient's body.

### DETAILED DESCRIPTION OF THE INVENTION

The present invention is directed to devices and methods for non-contact wound dressing for use with various types of wounds, and configured for application to various parts of the body as will be hereinafter described. In FIGS. 1-3, a first embodiment of the invention shows a wound dressing apparatus 10 having a body member 12 formed of a soft, flexible material such as a silicone rubber material or other suitable material, which is compatible for use in the invention. Depending on the environment in which the apparatus 10 is to be used, or the application in which it is used, the material can be modified to have predetermined characteristics. As an example, in an environment contaminated by biological, chemical, radioactive or other agents, the material from which apparatus 10 is made could be made impermeable to such agents. Alternatively, as desired environment may be created and maintained internal to the device 10. Additionally, by forming the body member 10 of a impermeable but flexible type of material, the body member is generally conformable to a particular body surface area, and less susceptible to stress applied thereto upon movement of the patient's body. The body member 12 comprises a base 14, forming a surface engaging portion, for application to the skin surface of a patient. In the embodiment shown, the base 14 is provided as a flange having a width to form a predetermined amount of surface area, which will contact the skin of a patient at a position adjacent a wound area. On a bottom surface of the base 14, an adhesive layer 16 is formed, being a silicone adhesive or other suitable adhesive material, which is compatible for use in adhering to the skin of a patient. Over the adhesive layer 16, a release liner 18 is provided to protect the adhesive until application to the skin is desired. For use, the release liner 18 is removed for exposure of the adhesive and application around the wound area. The base 14 provides a sealing edge for application of the body member body 12 to a surface, and is configured to have a shape and size to at least substantially circumscribe at least a portion of the area of a wound. To further facilitate application to a body portion, the base 14 may have one or more mounting tabs 23, which are not adhered to the skin by the adhesive layer. The tabs 23 may therefore be lifted from the skin surface and a securing mechanism positioned through holes 24 to facilitate mechanically attaching the base 14 to the patient, such as by suturing or other suitable method. Alternatively, tape or the like could be applied over tabs 23 and/or the flanges configuring the base 14, to facilitate retention on the body.

The body member 12 further comprises at least one wall 20 extending upwardly from the sealing edge or base 14, to form an enclosed space between the at least one upwardly extending wall and the surface to which the base 14 is applied. In the embodiment shown in FIGS. 1-3, the at least one wall extending upwardly from the base 14 is shown to comprise four sidewalls and a top wall in a generally rectangular configuration extending upward from the base 14. The wall 20 extends upwardly from the base 14 to form a dome-like structure over the space interior of the base 14 as best seen in FIG. 3, over the wound area in a non-contact configuration.

In addition, and as seen in FIGS. 1 and 2, the apparatus 10 may further comprise a layer of adhesive 26 formed on a top or other surface of the at least one upwardly extending wall 20. The adhesive layer 26 may again be covered with a release liner 28, to protect the adhesive layer until use is desired. In this configuration, the adhesive layer 26 provides the ability to adhere other materials to the exterior of the apparatus 10, for sealing about the apparatus 10. For many situations and/or environments, it may be desirable to apply a field dressing to a wound, to protect not only the wound from the external atmosphere and environment, but also to seal clothing at the area of the wound to reduce contamination through the clothing. In a military application, a soldier or other military personnel may wear a MOPP suit, to protect the wearer from biological or chemical agents, nuclear fallout, or other hazardous agents, which may be in the environment. Any breach in the MOPP suit can potentially expose the wearer to such agents. If a projectile or other object breaches the MOPP suit, and causes a wound, the apparatus 10 may be used to apply over the wound so as to isolate the wound from the external environment, after which the MOPP suit itself may be adhered to the adhesive layer 26 to reclose the breach in the suit made by the projectile or other object. In this manner, contamination from the environment through the breached MOPP suit may be substantially reduced. Even if no wound is present, the apparatus 10 could be used to reseal the suit in this manner.

As also seen in the embodiment of FIGS. 1-3, the dome-like structure created by the body 12 preferably includes a viewing window 30, which is disposed over the area of the wound encompassed by the dome-like structure, so as to enable a view of the wound through the apparatus 10 without requiring removal of the apparatus 10 or any alteration of the dressing. The viewing window 30 may be positioned only on a top portion of the dome-like structure, or the entire structure may be formed of a material, which is substantially transparent to allow viewing therethrough.

In this embodiment, it should be recognized that various aspects of the apparatus 10 may be suitably modified for a particular purpose, such as the use of a more rigid material in the manufacture of apparatus 10 or portions thereof, the use of opaque materials, or a configuration of the body 12 to form a dome-like structure over a desired area, but wherein the structure is differently configured than that shown in these figures. The body 12 could be formed in any of a variety of shapes or sizes, and the height of the dome-like structure relative to the surface on which the apparatus 10 is positioned can vary depending upon the particular application.

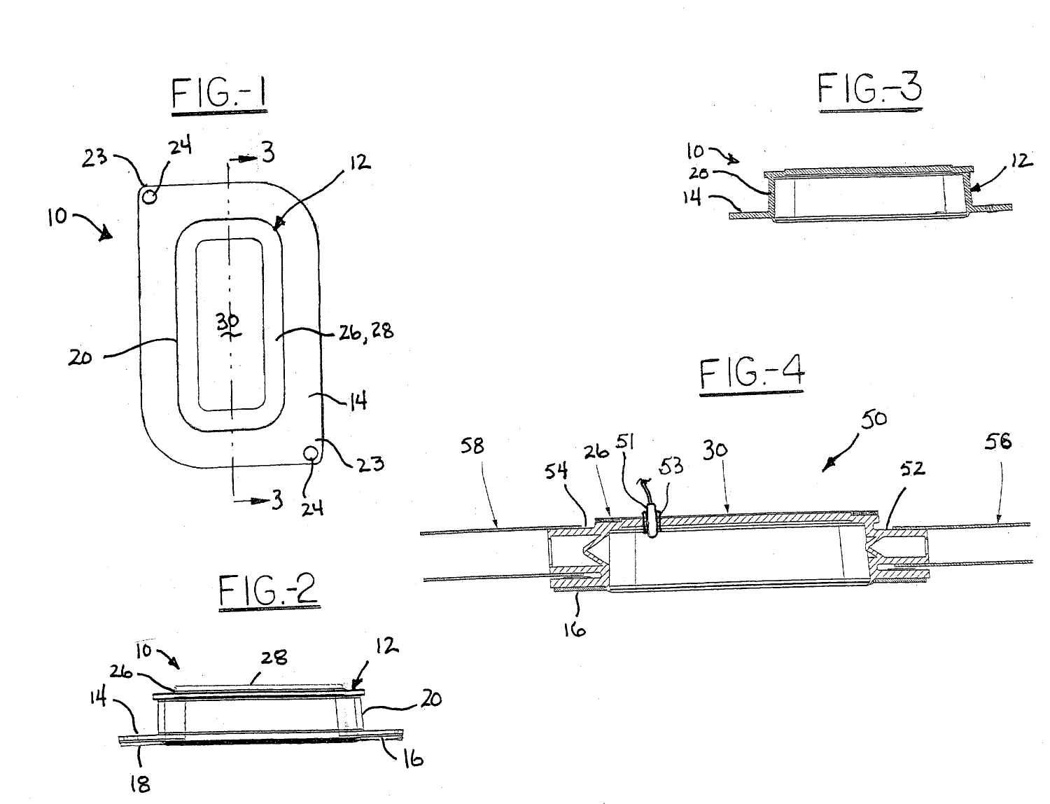

Turning to FIG. 4, another embodiment of the invention is shown, wherein the apparatus, generally designated 50, further comprises at least one access port, and as shown may comprise an inlet port 52 and an outlet port 54. Other structures associated with the apparatus 50 which are similar to the previous embodiment have retained similar reference numerals. Coupled to the inlet port 52, may be inlet tubing 56, such as flexible tubing used to deliver fluids to the apparatus 50. Similarly, an outlet tube 58 may be provided in association with the outlet 54, for the egress of fluids from the chamber created by the body member 12. As seen in FIG. 4, the outlet port 54 may be of a larger dimension than the inlet port 52 to facilitate the egress of fluids from the area of the wound under apparatus 50 if desired. Although this dimensional variation may be utilized, it should also be recognized that inlet and outlet ports of a similar size may also be used. The inlet and outlet tubes 56 and 58 may be configured to have a dimension to frictionally engage the outside or inside surface of the inlet and outlet ports 52 and 54 respectively, or could otherwise be operationally coupled to the ports in another suitable manner as would be apparent to a person of ordinary skill. The inlet port 52 and outlet port 54 may be used for introducing and/or removing fluids from the interior space of the apparatus 50, so as to treat a wound covered thereby. Any desired treatment fluid may be introduced for treatment of the wound in this manner, such as hyperberic oxygen, a thermally conditioned gas, such as for the application of heat to a wound, antibiotic flushes, saline solution, enzymatic debriding agents, haemostatic solutions or any other treatment fluid medium. Alternatively, other suitable access systems may be provided, and are contemplated. Any such access system can be referred to as a port. For example, the apparatus 10 or any portion thereof may be made of a resealing material, such as a polymer, which allows access to the enclosed area through a device, such as a needle, and minimizes exposure of the enclosed area to the external environment upon removal of the needle or the like, the material automatically reseals to protect the environment in the wound area. It is contemplated that use of this material on any portion of the apparatus 10 may provide an access port.

Further, to promote proper wound healing, it may be desirable to condition the environment created within the chamber situated about the wound under the dome-like structure provided by the apparatus according to the invention, wherein a conditioned atmosphere would be supplied through the inlet port 52 and removed via the outlet port 54 in a controlled manner. As an example, a multi-pressure pump (not shown) may be connected to the inlet tube 56 to provide a continuous, intermittent, or timed flow of hydrated air or fluids and/or hyperberic oxygen. The pump in connection with the desired treatment fluid would maintain the desired wound atmosphere within the chamber of apparatus 50. The desired atmosphere can be maintained by intermittent introduction of a desired treatment fluid, wherein it may be desirable to also introduce a probe 51 into the inlet port 52 and/or output port 54 to monitor the atmosphere within the chamber defined by the apparatus 50. Alternatively, such a monitoring probe could be inserted through a probe port 53 to continuously monitor the chamber atmosphere adjacent the wound, while still allowing the ingress and egress of treatment fluids via the inlet and outlet ports 52 and 54 respectively. In an example, a probe is positioned so as to be exposed to the space over the wound to measure desired parameters, such as moisture content, pH, temperature, fluid constituents or other variables. The probe could continuously monitor the environment and data communicated to a central database facility via wireless communication or the like. Alternatively, the probe could be interrogated to transmit data to a wand carried by a medial professional, to upload information as to the wound treatment. In this way, the size of wound, temperature, pH, and elements of byproducts produced by bacteria could be detected and communicated to be more readily accessible to a medical practitioner for evaluation of wound healing and refinement of a treatment process. If a wand is used to download information from a sensor, this information could be downloaded into a computer for medical record purposes instead of using real-time communications.

It may also be desirable to provide in association with the inlet port 52 and outlet port 54, an intermittent pumping action in association with the atmosphere within the chamber formed by apparatus 50 to promote stimulation of wound healing. It has been found that promoting circulation within the tissues of the wound by slight contraction using an intermittent suction action can promote wound healing. As an example, a vacuum pump (not shown) coupled to the apparatus 50 could provide intermittent suction at intervals to promote such wound healing in this manner.

The inlet and outlet ports 52 and 54 also would allow the apparatus 50 to be used for particular applications, such as pneumothorax or “sucking chest wounds”, wherein the integrity of the patient's chest wall is breached, and air is allowed to fill the pleural space, thereby eliminating the negative pressure needed to inflate the lung. The apparatus 50 could be used to seal the wound and permit emergency treatment in an ambulance or at the scene, including the application of suction to begin recreation of the negative pressure needed to fill the lung. The vacuum pump could again be connected via the ports to apparatus 50 for this purpose.

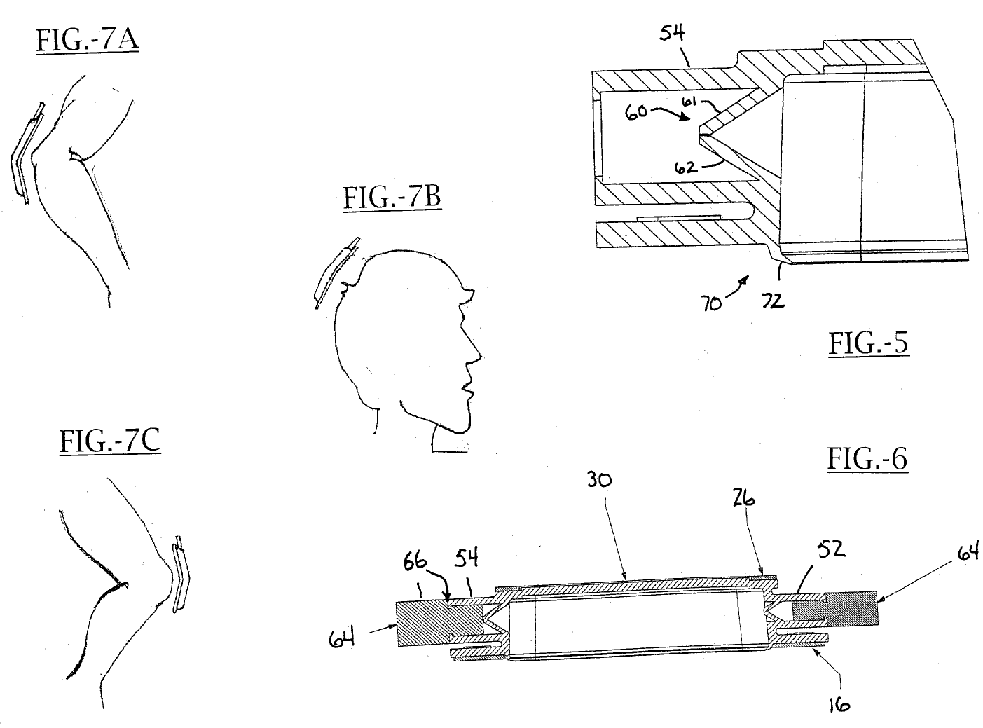

To facilitate use of the inlet and outlet ports 52 and 54, and with reference to FIG. 5, there may be provided a valve mechanism 60, which selectively closes the inlet and outlet ports 52 and 54 when not in use. Any type of suitable valve mechanism is contemplated, such as the self-sealing material mentioned previously, or other mechanical valves. The valve 60 may be provided as a self-closing bi-valve having members 61 and 62, which may be integrally formed in association with the inlet and outlet ports 52 and 54, or otherwise provided as desired. In the embodiment as shown in FIG. 5, the valve members 61 and 62 are formed in association with an output port 54, with the members 61 and 62 directed toward the outside of port 54, such that they will open upon pressure being applied from the interior chamber. As seen in FIG. 4, the inlet port 52 may be provided with a valve 60 having members 61 and 62 directed toward the interior of the chamber such that when pressure is supplied to introduce a treatment fluid or the like, the members 61 and 62 will open to allow ingress to the chamber. The valve 60 is configured to close the respective port in a normal “at rest” position. Upon the introduction of a fluid through the inlet port 52 via tubing 56 or the like, the valve 60 will be displaced to allow ingress through port 52 in the desired manner. Upon stopping flow of a fluid, the valve 60 would automatically return to its rest position to selectively close the port 52. In a similar manner, the valve 60 associated with an outlet port 54 would function to selectively be closed when no fluid flow is desired, and would be urged to an open position by fluid force to allow the egress of fluid from the apparatus 50 in the desired manner.

As also seen in FIG. 5, the apparatus according to the invention, such as shown in the embodiments described herein, may also include a secondary seal, generally designated 70, adapted to engage and seal with the supporting surface on which the apparatus is positioned, typically being the skin of a patient. The seal 70 is substantially formed as an outwardly extending lip 72, having a J-type configuration extending inwardly toward the chamber formed by the apparatus over the wound area. The secondary seal 70 in conjunction with the adhesive provided on base 14, provides a substantially fluid tight seal in association with the skin surface in a desired manner. Further, upon deformation of the body 12, the secondary seal configuration 70 tends to maintain the desired seal even upon motion of the body 14 relative to the skin surface. As an example, if the center portion of the dome-like construction formed by the apparatus is depressed, the base 14 may have an outward force exerted thereon, which could breach the integrity of the adhesive seal, but the secondary seal 70 maintains contact with the skin during any such movement.

As seen in FIG. 6, the inlet and/or outlet ports 52 and 54 may also be selectively closed when not in use by means of a plug 64 inserted into the port 52 and/or 54 to seal the port. A stop 66 may be formed in association with plug 64, which properly positions the plug 64 in the port 52 or 54. Alternatively, a stop could be formed on the interior of the port 52 and/or 54 to facilitate positioning of the plug member 62, and to properly seal the port in the desired manner.

The apparatus 10 and/or 50 according to the invention also facilitates wound treatment by use of light, such as low frequency infrared light, by allowing the transmission of such light through the viewing window or material from which the apparatus is made. It has been found that the incidence of this type of light upon a wound promotes and stimulates wound healing, and the characteristics of the apparatus according to the invention allow for such treatment procedures.

It should be recognized based upon the foregoing, that the apparatus according to the invention provides a closed wound dressing which is capable of providing sterile long term covering for a wound on the surface of the skin. The shape and material of the apparatus provide both a barrier to environmental pathogens and/or further trauma to the wound. Because the apparatus performs these functions in a non-contact configuration, the apparatus will prevent repetitive pressure injuries by cushioning the wound site. The shape of the apparatus desirably distributes forces of pressure over a relatively large footprint and away from the wound region to prevent pressure injuries and tissue ischemia over long periods of immobility. The ability to visualize a wound underlying the apparatus eliminates the need to perform painful and time consuming dressing changes, thus saving time and cost of discarded dressing material, as well as preventing possible contamination of the wound during such dressing changes. The apparatus also protects the medical practitioners as well as other patients and staff from contact with infected material and waste products from the wound. A predetermined atmosphere may be maintained in the wound environment, to stimulate and promote healing as desired. In addition to the treatment of typical wounds, the apparatus according to the invention may also be used to cover access ports to the body, such as arterio-venous, subclavian catheters, as well as other invasive devices, which may be used in the medical treatment of a patient.

Turning to FIG. 7, it should also be recognized that the apparatus according to the invention could be formed in a variety of configurations for use on a predetermined portion of the body. As merely examples, FIGS. 7A-C show the apparatus having a contoured body configuration for application to predetermined portions of a patient's body, such as the elbow as seen in FIG. 7A, the skull as seen in FIG. 7B, or the knee as seen in FIG. 7C. It should be recognized that other suitable body shapes may be formed having the desired attributes according to the invention, while conforming to a particular region of the patient. Further, for various applications, such as with scheduled surgical procedures, an apparatus according to the invention could be pre-made to provide a customized would dressing for use following the surgery. As previously indicated, the dimensions of the apparatus may be varied to accommodate particular wounds, such as for example to accommodate wounds incurred during surgical procedures, which may be rather thin but relatively long. Similarly, an appropriately dimensioned and configured apparatus according to the present invention could accommodate any other wound configuration.

The foregoing disclosure is illustrated of various embodiments of the present invention, but is not to be construed as limiting. Although several embodiments of the invention have been described, persons of ordinary skill in the art will readily appreciate that numerous modifications could be made without departing from the scope and spirit of the disclosed invention. As such, it should be understood that all such modifications are intended to be included within the scope of the invention as defined in the claims. Within the claims, any means-plus-function language is intended to cover the structure described in the present application as performing the recited function, and not only structural equivalence, but also equivalent structures. The written description and drawings illustrate the present invention and are not to be construed as limited to the specific embodiments disclosed. Modifications to the disclosed embodiments, as well as other embodiment's are included within the scope of the claims. The present invention is defined by the following claims, including equivalents thereof.

What is claimed is:

1. A wound treatment device comprising:
a housing configured to have a sealing edge for application to a surface, the sealing edge of a shape and size to at least substantially circumscribe the area of a wound, and at least one wall forming at least a substantially enclosed space between the at least one side wall and the surface to which the sealing edge is applied,
the sealing edge being adapted to be applied and retained on the surface to substantially seal a wound under the enclosed space from the external environment.

2. The wound treatment device as defined in claim 1, wherein the housing is made of a substantially soft and flexible material.

3. The wound treatment device as defined in claim 1, wherein said housing comprises a material substantially resistant to at least one predetermined agent within the external environment.

4. The wound treatment device as defined in claim 1, wherein said housing is at least partially made of a substantially transparent material.

5. The wound treatment device as defined in claim 1, wherein said sealing edge is substantially covered with an adhesive.

6. The wound treatment device as defined in claim 1, wherein said housing includes securing tabs for mechanically securing the housing to the surface.

7. The wound treatment device as defined in claim 1, wherein at least a portion of said at least one side wall is provided with adhesive on the exterior surface.

8. The wound treatment device as defined in claim 1, wherein said sealing edge has a contoured shape.

9. The wound treatment device as defined in claim 8, wherein said contoured shape substantially shaped for mating with a body surface.

10. The wound treatment device as defined in claim 1, wherein said housing includes at least one port.

11. The wound treatment device as defined in claim 10, wherein said device has an inlet port and an outlet port.

12. The wound treatment device as defined in claim 11, wherein said outlet port is larger than said inlet port.

13. The wound treatment device as defined in claim 10, wherein said at least one port includes a valve.

14. The wound treatment device as defined in claim 1, wherein said housing includes a secondary seal provided as a sealing lip which engages the surface.

15. The wound treatment device as defined in claim 1, wherein at least a portion of said housing comprises a self-sealing material through which devices may be selectively into the enclosed space.

16. A wound treatment device comprising:
a body member configured to have a sealing portion for application to a surface and at least one wall extending upwardly from the sealing portion to form at least a substantially enclosed space between the at least one side wall and the surface to which the sealing portion is applied,
the body member further comprising at least one port for introducing and/or removing a treatment fluid from the enclosed space, the at least one port being selectively closed to substantially seal the enclosed space from the external environment.

17. A wound treatment method comprising the steps of:
providing a dressing apparatus comprised of a body member configured to have a sealing portion for application to a skin surface and at least one wall extending upwardly from the sealing portion to form at least a substantially enclosed space, and at least one port selectively accessing the enclosed space,
applying the sealing portion to position the enclosed space over a wound under the at least one side wall,
selectively treating the wound through the dressing apparatus.

18. The wound treatment method as defined in claim 17, wherein at least one said at least one port includes a valve mechanism.

19. The wound treatment method as defined in claim 17, wherein the treating step comprises introducing treatment fluid into said enclosed space through said at least one port.

20. The wound treatment method as defined in claim 17, wherein the treating step comprises removing fluid from the enclosed space through said at least one port.

21. The wound treatment method as defined in claim 17, wherein the treating step comprises transmitting predetermined wavelengths of light through said body member onto a wound within said enclosed space.
